(19) United States
(12) Patent Application Publication
(10) Pub. No.: US 2015/0073544 A1
(43) Pub. Date: Mar. 12, 2015

(54) SUPERABSORBENT COATED STENTS FOR VASCULAR REDUCTION AND FOR ANCHORING VALVE REPLACEMENTS

(71) Applicants: The Trustees of The University of Pennsylvania, Philadelphia, PA (US); The Children's Hospital of Philadelphia, Philadelphia, PA (US)

(72) Inventors: Joseph H. Gorman, III, Lower Gwynedd, PA (US); Robert C. Gorman, Lower Gwynedd, PA (US); Matthew J. Gillespie, Brwyn Mawr, PA (US)

(21) Appl. No.: 14/382,783

(22) PCT Filed: Mar. 5, 2013
(86) PCT No.: PCT/US13/29034

§ 371 Date: Sep. 4, 2014

Related U.S. Application Data
(60) Provisional application No. 61/606,599, filed on Mar. 5, 2012.

(51) Int. Cl. A61F 2/24

(52) U.S. Cl. CPC A61F 2/2418
USPC 623/2.18

(57) ABSTRACT

A stent-based vascular reducer platform adapted for percutaneous delivery to and anchoring in a pulmonary or tricuspid valve annulus and a valved-stent prosthetic device adapted to be percutaneously delivered to the pulmonary or tricuspid valve annulus for mounting in the vascular reducer platform. The vascular reducer platform includes an at least partially self-expanding stent and a cuff made of an absorbent material disposed at least partially circumferentially around the outer and inner surfaces of the stent. Upon placement at an implantation site such as the pulmonary or tricuspid valve annulus, the absorbent material expands by absorption of a fluid to substantially adhere and seal the stent at the implantation site. Preferably, the cuff expansion is delayed for a time sufficient to permit positioning of the stent at the implantation site. The stent and cuff may also be used as a space-reducer for venous insufficiency, aortic aneurysms, and hydroureter treatment.

### CROSS-REFERENCE TO RELATED APPLICATIONS

The present patent application claims priority to U.S. Provisional Patent Application No. 61/606,599, filed Mar. 5, 2012. The content of that patent application is hereby incorporated by reference in its entirety.

### TECHNICAL FIELD

The present invention relates to stents for insertion into vascular spaces including veins and arteries as well as in the right ventricular outflow tract of the heart and, more particularly, to stents including superabsorbent material that slowly expands to the size of the dilated right ventricular outflow tract or blood vessel in which a stent is to be mounted, thereby facilitating anchoring of the stent and preventing leakage.

### BACKGROUND

Stents are widely used to treat diseased blood vessels and to help to anchor valve replacements in the heart. However, such stents have limited use in large or dilated blood vessels or in large areas such as the right ventricular outflow tract where chronic regurgitation results in dilation and distortion of the target area to the point where it is too large to allow proper mounting of conventional valved stents. This makes it very difficult to mount replacement pulmonary or tricuspid valves using stents as the anchoring mechanism. Also, since the stents are typically uniform in shape, they may permit leakage around the stent due to non-uniformities at the mounting site. A vascular reduction mechanism is desired that permits the stent to mount more firmly in dilated vascular spaces (such as veins, arteries, valve annuli) and to prevent leakage around the stent. The present invention addresses these needs in the art.

### SUMMARY

The present inventors have addressed the above needs in the art by providing a stent-based platform adapted for percutaneous delivery to and anchoring in a pulmonary or tricuspid valve annulus. Implantation of this platform creates a uniform landing zone for subsequent percutaneously delivered valved-stent implantation into the pulmonary or tricuspid valve annulus. The platform includes an at least partially self-expanding stent and a cuff comprising an absorbent material disposed at least partially circumferentially around the outer and inner surfaces of the stent-based platform. Upon placement at an implantation site such as the pulmonary or tricuspid valve annulus, the absorbent material expands by absorption of a fluid to create a substantial seal between the vessel wall/annulus and the outer portion of the stent. The inner layer of superabsorbent material will expand to create a zone that is uniform in diameter and shape, thereby creating a customizable landing zone for subsequent valved-stent implantation. Preferably, the expansion of the superabsorbent material is delayed for a time sufficient to permit positioning of the stent at the implantation site.

The above needs in the art are also met by providing a vascular reducer comprising an at least partially self-expanding stent and a sealing cuff comprising an absorbent material disposed at least partially circumferentially around the outer and inner surface of the stent. Upon installation in the blood vessel, the absorbent material expands by absorption of a fluid to substantially adhere the stent at an implantation site. The cuff expansion is preferably delayed for a time sufficient to permit positioning of the stent at the implantation site.

In exemplary embodiments, the absorbent material is implemented as a bi-layer system in which an outer layer is a hydrophobic material that prevents the absorbent material from expanding while the stent is being placed. The outer layer is also adapted to elute off slowly to allow underlying absorbent material to expand to permit positioning of the stent. The absorbent material may be based on poly(acrylic acid) that is cross-linked with poly(ethylene glycol) diacrylate where the swelling volume and softness of the absorbent material is varied by manipulating the extent of cross-linking. Also, the hydrophobic material may comprise a water proof polymer barrier deposited on the absorbent material using initiated chemical vapor deposition.

Embodiments within the scope of the invention as described in the following detailed description and claimed in the attached claims.

### BRIEF DESCRIPTION OF THE DRAWINGS

The various novel aspects of the invention will be apparent from the following detailed description of the invention taken in conjunction with the accompanying drawings, of which:

FIG. 1 illustrates a vascular stent that is covered on both the inside and outside with a cuff of superabsorbent (SA) material.

FIG. 2 illustrates the device of FIG. 1 compressed for insertion into an angioplasty balloon for delivery to the implantation site.

FIG. 3 illustrates the compressed vascular stent of FIG. 2 prior to deployment at the implantation site.

FIG. 4 illustrates the deployed device of FIG. 1 where the superabsorbent material has expanded in a pulmonary annulus for anchoring.

FIG. 5 illustrates the hour-glass shape formed by the expanded internal cuff of the device of FIG. 1.

FIG. 6 illustrates the deployed device of FIG. 1 with a deployed valved stent mounted therein.

### DETAILED DESCRIPTION OF ILLUSTRATIVE EMBODIMENTS

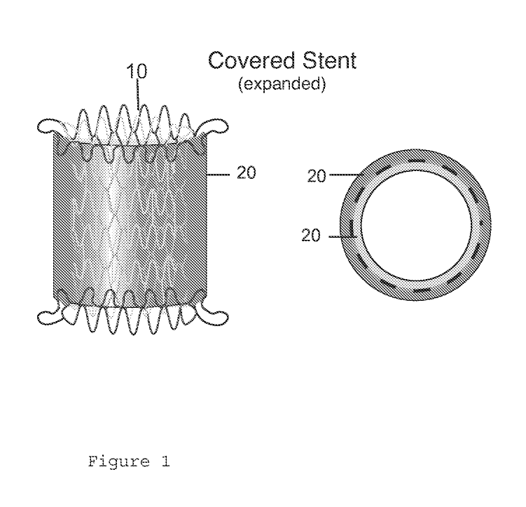

The invention will be described in detail below with reference to FIGS. 1-6. Those skilled in the art will appreciate that the description given herein with respect to those figures is for exemplary purposes only and is not intended in any way to limit the scope of the invention. All questions regarding the scope of the invention may be resolved by referring to the appended claims.

The invention broadly addresses the problem of deploying devices in dilated body lumens and/or in large cavities by reducing the diameter to an appropriate and uniform size/dimension for subsequent implantation of a stent, a valved stent, or some other device. The superabsorbent coated stent can also provide a platform for accepting a pulmonary or tricuspid valve prosthesis.

In accordance with this embodiment of the invention, a successful percutaneously placed “vascular reducer” has the following characteristics:
1. Deliverable using established stenting techniques;
2. Low profile for delivery via standard vascular sheath sizes;
3. Upon expansion, it should be large enough in diameter for secure deployment into dilated right ventricle outflow tracts; and
4. Provide an appropriate “landing zone” (“hourglass shaped”) for subsequent deployment and secure anchoring of a pulmonary valved-stent.

A device meeting these requirements is shown in FIG. 1. The device of FIG. 1 includes a vascular stent 10 (e.g., stainless steel, Cobalt, chromium or Nitinol) covered on both the inside and outside with a cuff 20 of superabsorbent (SA) material. The interior covering of the stent is a tailored cuff filled with superabsorbent material that is engineered to blossom into a firm, hourglass shape upon expansion; making the lumen of the stent circumferentially uniform. This hourglass shaped lumen serves as an ideal landing site for valved-stent deployment and anchoring.

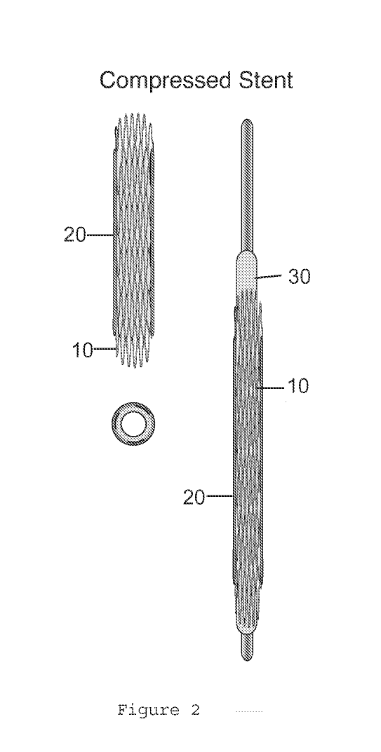

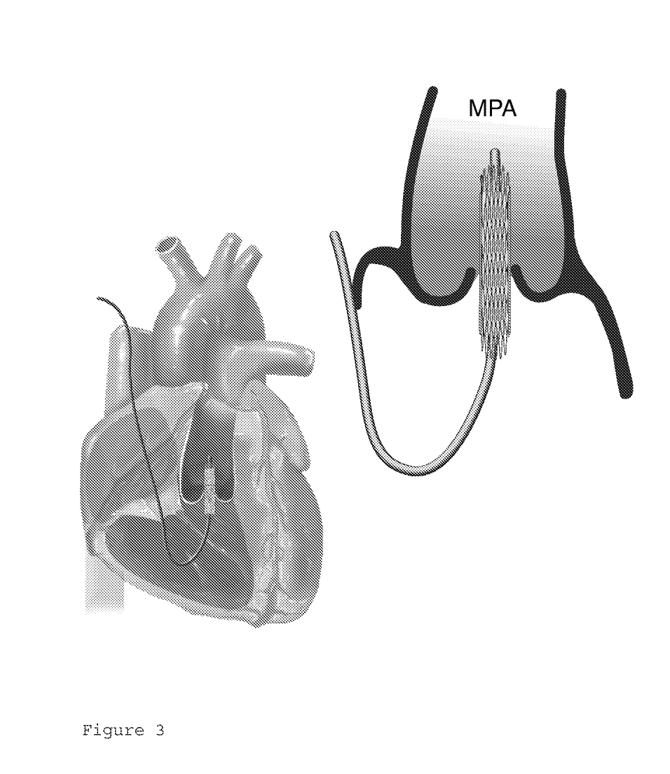

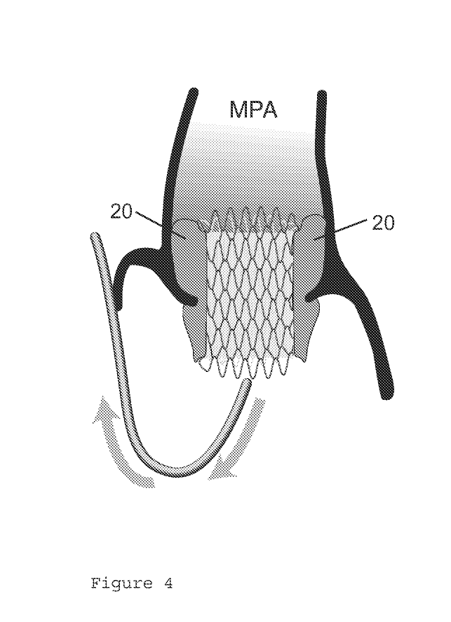

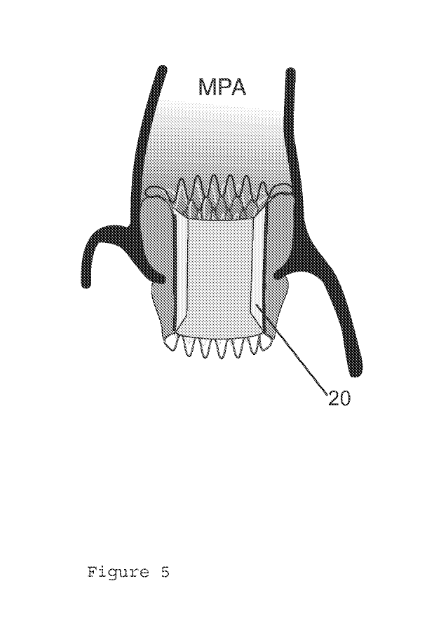

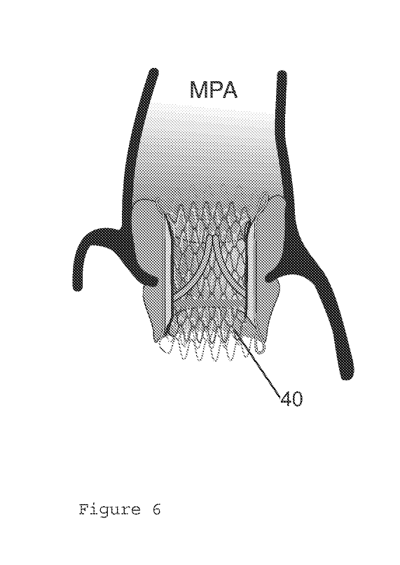

As shown in FIG. 2, the device of FIG. 1 compresses for insertion into an angioplasty balloon 30 for delivery, for example, to a pulmonary valve annulus (FIG. 3). FIG. 4 shows the device once the superabsorbent material begins to expand in the pulmonary valve annulus. As illustrated, the superabsorbent material of the cuff 20 expands to fit the contours of the mounting site. FIG. 5 illustrates the hour-glass shape formed by the expanded superabsorbent cuff 20, and FIG. 6 illustrates the device with a deployed valved stent 40 mounted therein.

The outside covering of the cuff 20 on the stent 10 expands when the stent is deployed into the deployment position, for example, the right ventricle outflow tract (RVOT). This outer covering of the cuff 20 serves to fill in potential gaps between the stent 10 and the vascular walls thus improving apposition and preventing leakage around the stent 10. The superabsorbent material is preferably implemented as a bi-layer system in which the outer layer is a hydrophobic material that will prevent the super absorbent material from expanding while the stent 10 is being placed. This layer is designed to elute off slowly to ultimately allow the underlying superabsorbent material to expand. In this way, a low profile covered stent 10 can be advanced through the vasculature and then deployed using standard stenting techniques. The device will then “morph” into its predetermined shape as the SA material expands to fill the vascular space.

In an exemplary embodiment, the super-absorbent material best suited for this application is based on poly(acrylic acid) that is cross-linked with poly(ethylene glycol) diacrylate. The cross-linking can be controlled by a photopolymerization mechanism. The overall swelling volume and softness can be optimized by manipulating the extent of cross-linking of the material. In addition, the expansion of the superabsorbent material needs to be delayed while the device is being placed. To achieve this, the hydrophobic outer layer in an exemplary embodiment incorporates a coating technology called initiated chemical vapor deposition (iCVD) to place a water proof polymer barrier on the surface of the cuff fabric. iCVD is a proven technique that can synthesize and integrate polymer coatings on complex 3D structures like the cuff fabric. The polymer is based on copolymerization of hydrophilic (N-vinyl-2-pyrrolidone/methacrylic acid) and hydrophobic (alkyl acrylate) components that are designed to harmlessly elute off after a given amount of time thus allowing the super absorbent material to expand and seal. The water absorption delay can be engineered by manipulating the structure and properties of the coating polymer through control of iCVD processing, and will be dictated by device placement time which should be approximately 30 minutes.

Those skilled in the art will appreciate that the invention is particularly useful in specific circumstances where vascular occlusion and/or flow reduction is necessary (e.g., pulmonary artery banding, PDA occlusion, or aneurysm/pseudoaneurysm treatment). However, the device of this embodiment also has multiple applications for patients with pulmonary valve incompetence following surgical and/or catheter-based intervention. The majority of these patients would benefit from pulmonary valve replacement; however, most are not eligible for the minimally invasive approach due to the limitations in the current technology. Surgical valve replacement is an option, but it is associated with significant morbidity. Thus, definitive therapy is often delayed subjecting patients to chronic pulmonary insufficiency, leaving them at risk for right ventricular failure. This embodiment can be used to limit such valve incompetence.

Those skilled in the art will further appreciate that the invention has several advantages. Firstly, it can be implanted into the RVOT using standard stenting techniques, with which all interventional cardiologists are familiar. Secondly, the device maintains a low profile for percutaneous delivery, minimizing vascular trauma. Thirdly, the engineered superabsorbent cuff forms the ideal landing zone (size, shape and firmness) for percutaneous valved-stent implantation. Other advantages will be apparent to those skilled in the art.

Those skilled in the art will also appreciate that the invention may be applied to other applications and may be modified without departing from the scope of the invention. For example, those skilled in the art will appreciate that the devices and techniques of the invention may be used to replace the tricuspid valve as well as the pulmonary valve. Also, the devices and techniques of the invention may be used to provide a vascular reducer when a reduced vessel diameter is desired, as when mounting a stent. Accordingly, the scope of the invention is not intended to be limited to the exemplary embodiments described above, but only by the appended claims.

1. A vascular reducer comprising:
a stent-based platform adapted for percutaneous delivery and anchoring in a heart valve annulus, said platform comprising an at least partially self-expanding stent and a cuff comprised of an absorbent material disposed at least partially circumferentially around the outer and inner surface of said stent, wherein said absorbent material expands by absorption of a fluid to substantially seal said stent at an implantation site, and wherein said expansion of said absorbent material is delayed for a time sufficient to permit positioning of said stent at said implantation site; and
a valved-stent prosthetic device adapted to be percutaneously delivered to the heart valve annulus for mounting in said platform.

2. The vascular reducer according to claim 1, wherein the platform is adapted for anchoring in a pulmonary valve annulus and the valved-stent prosthetic device comprises a pulmonary valved-stent prosthetic device adapted for delivery to the pulmonary valve annulus.

3. The vascular reducer according to claim 1, wherein the platform is adapted for anchoring in a tricuspid valve annulus and the valved-stent prosthetic device comprises a tricuspid valved-stent prosthetic device adapted for delivery to the tricuspid valve annulus.

4. The vascular reducer according to claim 1, wherein the absorbent material is implemented as a bi-layer system in which an outer layer is a hydrophobic material that prevents the absorbent material from expanding while the stent is being placed, said outer layer adapted to elute off slowly to allow underlying absorbent material to expand to permit positioning of said stent.

5. The vascular reducer according to claim 4, wherein the absorbent material is based on poly(acrylic acid) that is cross-linked with poly(ethylene glycol) diacrylate and the swelling volume and softness of the absorbent material is varied by manipulating the extent of cross-linking.

6. The vascular reducer according to 4, wherein the hydrophobic material comprises a water proof polymer barrier deposited on the absorbent material using initiated chemical vapor deposition.

7. A vascular reducer comprising an at least partially self-expanding stent and a sealing cuff comprising an absorbent material disposed at least partially circumferentially around the outer and inner surface of said stent, wherein said absorbent material expands by absorption of a fluid to substantially adhere and seal said stent at an implantation site, and wherein said expansion of said absorbent material is delayed for a time sufficient to permit positioning of said stent at said implantation site.

8. The vascular reducer according to claim 7, wherein the absorbent material is implemented as a bi-layer system in which an outer layer is a hydrophobic material that prevents the absorbent material from expanding while the stent is being placed, said outer layer adapted to elute off slowly to allow underlying absorbent material to expand to permit positioning of said stent.

9. The vascular reducer according to claim 8, wherein the absorbent material is based on poly(acrylic acid) that is cross-linked with poly(ethylene glycol) diacrylate and the swelling volume and softness of the absorbent material is varied by manipulating the extent of cross-linking.

10. The vascular reducer according to claim 8, wherein the hydrophobic material comprises a water proof polymer barrier deposited on the absorbent material using initiated chemical vapor deposition.

11. A vascular reducer comprising:
a stent-based platform adapted for percutaneous delivery and anchoring in a heart valve annulus, said platform comprising an at least partially self-expanding stent and a cuff comprised of an absorbent material comprising poly(acrylic acid) that is cross-linked with poly(ethylene glycol) diacrylate that disposed at least partially circumferentially around the outer surface of said stent, wherein said absorbent material expands by absorption of a fluid to substantially seal said stent at an implantation site, and wherein said expansion of said absorbent material is delayed for a time sufficient to permit positioning of said stent at said implantation site; and
a valved-stent prosthetic device adapted to be percutaneously delivered to the heart valve annulus for mounting in said platform.

12. The vascular reducer according to claim 11, wherein the platform is adapted for anchoring in a pulmonary valve annulus and the valved-stent prosthetic device comprises a pulmonary valved-stent prosthetic device adapted for delivery to the pulmonary valve annulus.

13. The vascular reducer according to claim 11, wherein the platform is adapted for anchoring in a tricuspid valve annulus and the valved-stent prosthetic device comprises a tricuspid valved-stent prosthetic device adapted for delivery to the tricuspid valve annulus.

14. The vascular reducer according to claim 11, wherein the absorbent material is implemented as a bi-layer system in which an outer layer is a hydrophobic material that prevents the absorbent material from expanding while the stent is being placed, said outer layer adapted to elute off slowly to allow underlying absorbent material to expand to permit positioning of said stent.

15. The vascular reducer according to claim 14, wherein the hydrophobic material comprises a water proof polymer barrier deposited on the absorbent material using initiated chemical vapor deposition.

16. The vascular reducer according to claim 14, wherein the hydrophobic material is based on the copolymerization of hydrophilic and hydrophobic components.

17. The vascular reducer according to claim 16, wherein the hydrophilic component is N-vinyl-2-pyrrolidone/methyacrylic acid and the hydrophobic component is an alkyl acrylate.
